(19) United States
(12) Patent Application Publication
(10) Pub. No.: US 2021/0226129 A1
(43) Pub. Date: Jul. 22, 2021

(54) CHARGE TRANSPORT MATERIAL, INK COMPOSITION USING SAID MATERIAL, ORGANIC ELECTRONIC ELEMENT, ORGANIC ELECTROLUMINESCENT ELEMENT, DISPLAY ELEMENT, LIGHTING DEVICE AND DISPLAY DEVICE

(71) Applicant: HITACHI CHEMICAL COMPANY, LTD., Tokyo (JP)

(72) Inventors: Kazuyuki KAMO, Tsukuba-shi (JP); Naoki ASANO, Tsukuba-shi (JP); Hiroshi TAKAIRA, Hitachinaka-shi (JP)

(73) Assignee: Hitachi Chemical Company, Ltd., Tokyo (JP)

(21) Appl. No.: 16/093,713

(22) PCT Filed: Apr. 13, 2017
(86) PCT No.: PCT/JP2017/015154

§ 371 Date: Oct. 15, 2018

(30) Foreign Application Priority Data
Apr. 15, 2016 (JP) 2016-082194

(51) Int. Cl. H01L 51/00; C08G 61/12; C09D 11/52

(52) U.S. Cl. CPC H01L 51/0035; C08G 61/122; C08G 61/121; C09D 11/52; C08G 2261/124; C08G 2261/148; H01L 51/5012; C08G 2261/3245; C08G 2261/411; C08G 2261/512; C08G 2261/91; H01L 2251/5338; C08G 2261/3162

(57) ABSTRACT

A charge transport polymer containing a structural unit having an N-aryl phenoxazine skeleton is produced, and is used as a charge transport material.

### TECHNICAL FIELD

The present invention relates to a charge transport material, and an ink composition that uses the material. Further, the present invention also relates to an organic electronic element, an organic electroluminescent element, a display element, a lighting device and a display device, each having an organic layer that uses the above charge transport material or the above ink composition.

### BACKGROUND ART

Organic electronic elements are elements which use an organic substance to perform an electrical operation, and because they are expected to be capable of providing advantages such as low energy consumption, low prices and superior flexibility, they are attracting considerable attention as a potential alternative technology to conventional inorganic semiconductors containing mainly silicon.

Examples of organic electronic elements include organic electroluminescent elements (hereafter also referred to as “organic EL elements”), organic photoelectric conversion elements, and organic transistors and the like.

Among organic electronic elements, organic EL elements are attracting attention for potential use in large-surface area solid state lighting source applications to replace incandescent lamps and gas-filled lamps and the like. Further, organic EL elements are also attracting attention as the leading self-luminous display for replacing liquid crystal displays (LCD) in the field of flat panel displays (FPD), and commercial products are becoming increasingly available.

Depending on the organic materials used, organic EL elements are broadly classified into low-molecular weight type organic EL elements and polymer type organic EL elements. In polymer type organic EL elements, polymer compounds are used as the organic materials, whereas in low molecular weight type organic EL elements, low-molecular weight materials are used. Compared with low-molecular weight type organic EL elements in which film formation is mainly performed in vacuum systems, polymer type organic EL elements enable simple film formation to be performed by wet processes such as printing or inkjet application, and are therefore expected to be essential elements in future large-screen organic EL displays.

Accordingly, the development of materials that are suited to wet processes is being actively pursued, and investigations such as those disclosed in Patent Document 1 are being undertaken.

### PRIOR ART DOCUMENTS

### Patent Document

Patent Document 1: JP 2006-279007 A

### DISCLOSURE OF INVENTION

### Problems Invention Aims to Solve

Generally, organic EL elements produced by wet processes using polymer compounds have the advantages that cost reductions and surface area increases can be achieved with relative ease. However, organic EL elements containing thin films produced using conventional polymer compounds still require further improvements in terms of organic EL element characteristics including the drive voltage, the emission efficiency and the emission lifespan.

In light of the above circumstances, the present invention has the objects of providing a charge transport material containing a polymer compound that can be used in an organic electronic element, and an ink composition that contains the material. Further, the present invention also has the objects of using the above charge transport material or ink composition to provide an organic electronic element and an organic EL element having excellent drive voltage, emission efficiency and emission lifespan characteristics, as well as providing a display device, a lighting device and a display device that use the organic EL element.

### Means for Solution of the Problems

As a result of intensive investigation, the inventors of the present invention discovered that a charge transport polymer having a specific structural unit was ideal as a charge transport material for forming an organic layer of an organic electronic element, and they were therefore able to complete the present invention. Embodiments of the present invention are described below, but the present invention is not limited to these embodiments.

One embodiment relates to a charge transport material containing a charge transport polymer, wherein the charge transport polymer contains a structural unit having an N-aryl phenoxazine skeleton.

The above structural unit having an N-aryl phenoxazine skeleton preferably includes at least one structural unit selected from the group consisting of divalent structural units L1 and trivalent or higher structural units B1.

The charge transport polymer described above preferably also includes at least one structural unit, besides the above structural unit having an N-aryl phenoxazine skeleton, selected from the group consisting of divalent structural units L2 having charge transport properties and trivalent or higher structural units B2 having charge transport properties.

It is more preferable that the charge transport polymer described above also includes a divalent structural unit L2 having charge transport properties besides the above structural unit having an N-aryl phenoxazine skeleton. This divalent structural unit L2 having charge transport properties preferably contains at least one structure selected from the group consisting of aromatic amine structures, carbazole structures, thiophene structures, benzene structures and fluorene structures. The charge transport polymer described above preferably has a structure that branches in three or more directions. The charge transport material described above is preferably used as a hole injection material.

Another embodiment relates to an ink composition containing the charge transport material of the embodiment described above and a solvent.

Another embodiment relates to an organic electronic element having an organic layer formed using the charge transport material of the embodiment described above or the ink composition of the embodiment described above.

Another embodiment relates to an organic electroluminescent element having an organic layer formed using the charge transport material of the embodiment described above or the ink composition of the embodiment described above. The organic electroluminescent element preferably also has a flexible substrate, and the flexible substrate preferably includes a resin film.

Another embodiment relates to a display element having the organic electroluminescent element of the embodiment described above.

Another embodiment relates to a lighting device having the organic electroluminescent element of the embodiment described above.

Another embodiment relates to a display device having the lighting device of the embodiment described above, and a liquid crystal element as a display unit.

### Effects of the Invention

The present invention is able to provide an organic electronic element and an organic EL element having a low drive voltage and excellent emission efficiency and emission lifespan, and can also provide a display element, a lighting device and a display device that use the organic EL element.

### BRIEF DESCRIPTION OF THE DRAWINGS

FIG. 1 is a schematic cross-sectional view illustrating one embodiment of an organic EL element.

### EMBODIMENTS FOR CARRYING OUT THE INVENTION

Embodiments of the present invention are described below in further detail. However, the present invention is not limited to the following embodiments.

### <Charge Transport Material>

The charge transport material contains a charge transport polymer, and the charge transport polymer contains a structural unit having an N-aryl phenoxazine skeleton. The charge transport material may contain one type, or two or more types, of the above charge transport polymer. The charge transport polymer is described below in further detail.

### (Charge Transport Polymer)

The charge transport polymer disclosed in this description may be any polymer that displays charge transport properties, and contains a structural unit having an N-aryl phenoxazine skeleton within the molecule. The charge transport polymer containing the structural unit having an N-aryl phenoxazine skeleton may have a linear structure or a branched structure. The charge transport polymer preferably contains at least a divalent structural unit L having charge transport properties and a monovalent structural unit T that constitutes the terminal portions, and may also contain a trivalent or higher structural unit B that forms a branched portion. The charge transport polymer may have only one type of each of these structural units, or may contain a plurality of types of each structural unit. In the charge transport polymer, the various structural units are bonded together at “monovalent” to “trivalent or higher” bonding sites.

In the above charge transport polymer, at least one of the structural units L, T and B has an N-aryl phenoxazine skeleton. In other words, the charge transport polymer contains at least a monovalent or higher structural unit having an N-aryl phenoxazine skeleton.

### (Structural Unit Having N-Aryl Phenoxazine Skeleton)

As illustrated in the formula below, an “N-aryl phenoxazine skeleton” means a structure in which a substituted or unsubstituted aryl group (Ar) is bonded to the N atom of a phenoxazine skeleton. The aromatic rings in the phenoxazine skeleton may be unsubstituted, or may have a substituent R. In the formula below, 1 represents an integer of 0 to 4, and indicates the number of substituents R. The substituent R is the same as R in a structural unit AF described below.

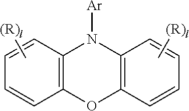

The “structural unit having an N-aryl phenoxazine skeleton” means a structural unit that includes an atom grouping in which at least one hydrogen atom has been removed from the N-aryl phenoxazine skeleton described above. In the charge transport polymer, the monovalent or higher structural unit having an N-aryl phenoxazine skeleton (hereafter also referred to as the “structural unit AF”) is bonded to one or more other structural units at one or more bonding sites.

In one embodiment, the structural unit AF may be at least one of a monovalent, divalent or trivalent or higher structural unit derived from an N-aryl phenoxazine skeleton. In another embodiment, the structural unit AF may have at least one monovalent group (structural unit) having an N-aryl phenoxazine skeleton as a substituent on a portion of the main skeleton that forms a structural unit. By including the structural unit AF in the charge transport polymer, characteristics of an organic EL element such as the drive voltage, the emission efficiency and the emission lifespan can be easily improved. From the viewpoints of the ease of compound synthesis and the durability of the organic EL element, the structural unit AF is preferably not higher than hexavalent, and is more preferably tetravalent or lower.

The structural unit AF is described below in further detail.

### (Monovalent Structural Unit AF)

A monovalent structural unit AF has an N-aryl phenoxazine skeleton, and has one bonding site with another structural unit. In one embodiment, the monovalent structural unit AF preferably has a structure in which one hydrogen atom has been removed from an N-aryl phenoxazine skeleton. This embodiment includes structures in which the hydrogen atom has been removed from a substituent on the N-aryl phenoxazine skeleton.

Specific examples of the monovalent structural unit AF are shown below. In one embodiment, the charge transport polymer preferably includes a structural unit shown below as a monovalent structural unit T1 having charge transport properties.

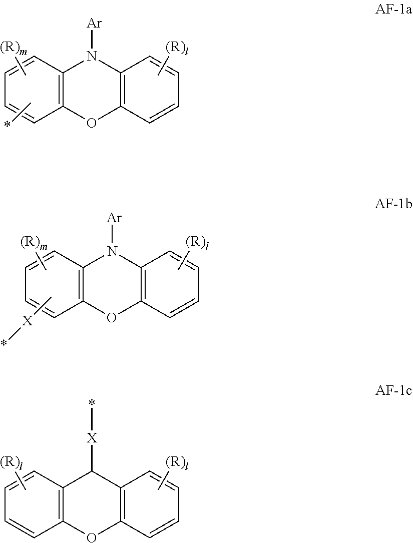

In the above structural units, 1 represents an integer of 0 to 4 and m represents an integer of 0 to 3, with each representing a number of R groups. The symbol “*” represents a bonding site with another structural unit. In one embodiment, each R is, independently, selected from the group consisting of linear, cyclic or branched alkyl groups, alkenyl groups, alkynyl groups and alkoxy groups of 1 to 22 carbon atoms, and aryl groups and heteroaryl groups of 2 to 30 carbon atoms. The aryl groups and heteroaryl groups may have an additional substituent R1. This additional substituent R1 in the aryl groups and heteroaryl groups is preferably a linear, cyclic or branched alkyl group of 1 to 22 carbon atoms.

In the above structural unit, R is preferably a substituted or unsubstituted aryl group of 6 to 30 carbon atoms, is more preferably a substituted or unsubstituted aryl group of 6 to 20 carbon atoms, and is even more preferably a substituted or unsubstituted phenyl group or naphthyl group. In one embodiment, when the charge transport polymer has a polymerizable functional group at a terminal portion, at least one R may be a group having a polymerizable functional group.

In the structural unit described above, Ar is an atom grouping in which one hydrogen atom has been removed from an aromatic hydrocarbon. Here, the aromatic hydrocarbon may have a structure in which two or more aromatic rings are bonded together, such as biphenyl, or may have a structure in which two or more aromatic rings are condensed, such as naphthalene. More specifically, Ar is a substituted or unsubstituted aryl group of 6 to 30 carbon atoms. The substituents on the aryl group may be the same as the additional substituent R1 described above. Ar is more preferably a substituted or unsubstituted aryl group of 6 to 20 carbon atoms, and is even more preferably a substituted or unsubstituted phenyl group or naphthyl group.

In the structural unit shown above, X represents a divalent linking group, and is an atom grouping in which two hydrogen atoms have been removed from an aromatic hydrocarbon. In other words, X may be an atom grouping in which one hydrogen atom has been removed from an Ar group described above. More specifically, X is preferably a substituted or unsubstituted arylene group of 6 to 30 carbon atoms, and is more preferably a substituted or unsubstituted arylene group of 6 to 20 carbon atoms. X is preferably a substituted or unsubstituted phenylene group or naphthylene group, and is more preferably a phenylene group. The phenylene group may be a 1,2-phenylene group, 1,3-phenylene group or 1,4-phenylene group, but is preferably a 1,4-phenylene group.

Specific examples of preferred monovalent structural units AF are shown below. However, the monovalent structural unit AF is not limited to the following structural units.

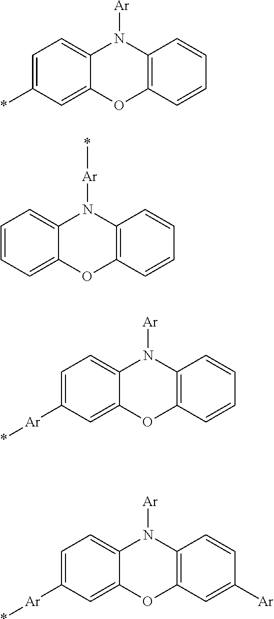

In the formulas, each Ar represents an aforementioned substituted or unsubstituted aryl group or arylene group of 6 to 30 carbon atoms. The symbol “*” represents a bonding site with another structural unit.

### (Divalent Structural Unit AF)

A divalent structural unit AF has an N-aryl phenoxazine skeleton, and has two bonding sites with other structural units. In one embodiment, the divalent structural unit AF preferably has a structure in which two hydrogen atoms have been removed from an N-aryl phenoxazine skeleton. This embodiment includes structures in which a hydrogen atom has been removed from a substituent on the N-aryl phenoxazine skeleton.

Specific examples of the divalent structural unit AF are shown below. In one embodiment, the charge transport polymer preferably includes a structural unit shown below as a divalent structural unit L1 having charge transport properties.

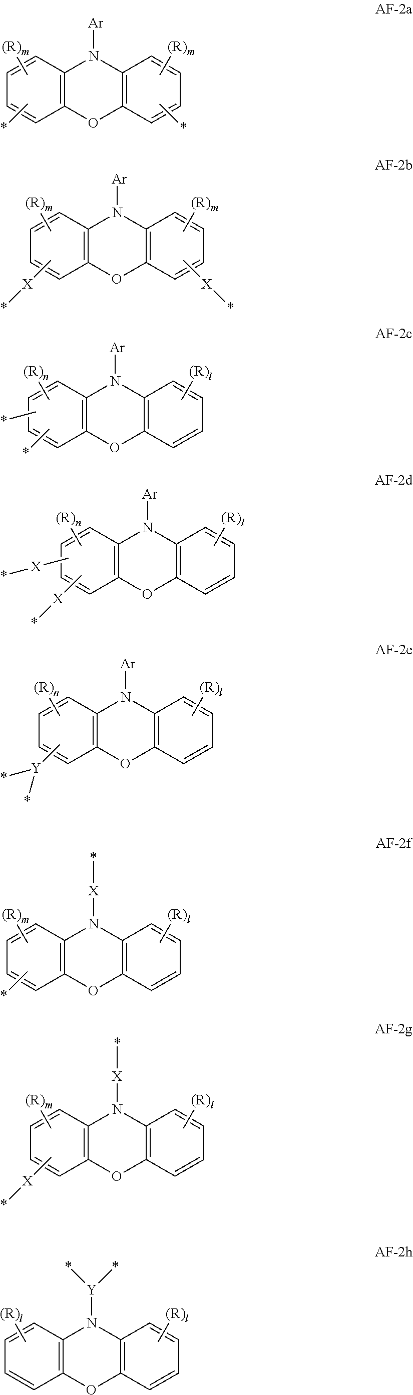

In the above structural units, 1 represents an integer of 0 to 4, m represents an integer of 0 to 3 and n represents an integer of 0 to 2, with each representing a number of R groups. The symbol “*” represents a bonding site with another structural unit. R, Ar and X are the same as described above in relation to the monovalent structural unit AF.

In the above structural units, Y represents a trivalent linking group, and is an atom grouping in which three hydrogen atoms have been removed from an aromatic hydrocarbon. In other words, Y may be an atom grouping in which two hydrogen atoms have been removed from an Ar group described above. More specifically, Y is preferably a substituted or unsubstituted arenetriyl group of 6 to 30 carbon atoms, and is more preferably a substituted or unsubstituted arenetriyl group of 6 to 20 carbon atoms.

Specific examples of preferred divalent structural units AF are shown below. However, the divalent structural unit AF is not limited to the following structural units.

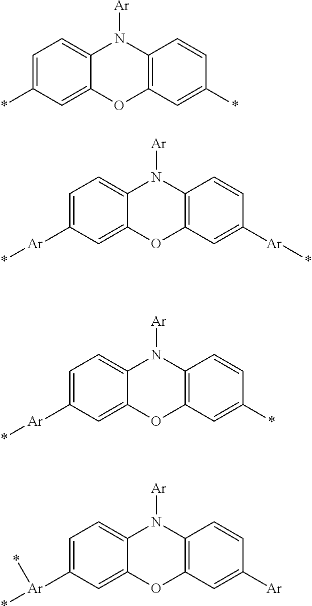

In the formulas, each Ar represents an aforementioned substituted or unsubstituted aryl group, arylene group or arenetriyl group of 6 to 30 carbon atoms. The symbol “*” represents a bonding site with another structural unit.

Specific examples of even more preferred divalent structural units AF are shown below. However, the divalent structural unit AF is not limited to the following structural units. In the following formulas, each Ar represents an aforementioned substituted or unsubstituted aryl group of 6 to 30 carbon atoms.

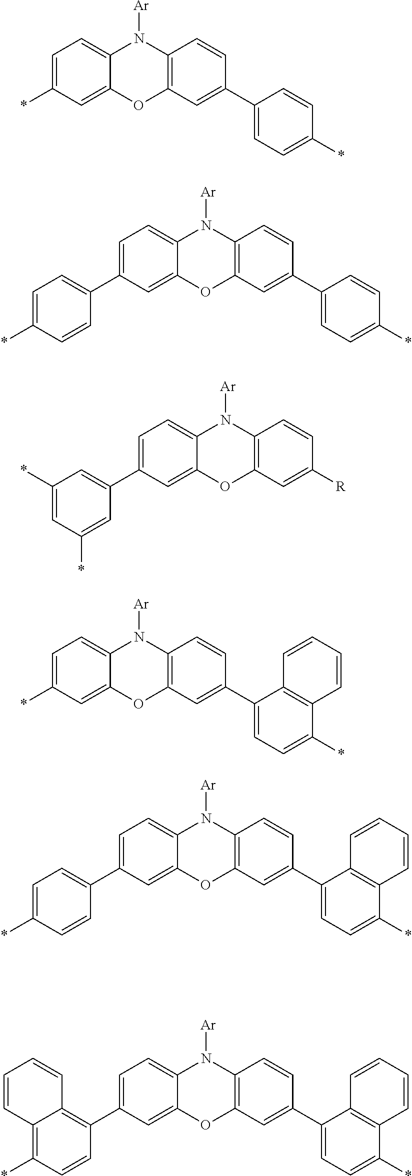

In another embodiment, the divalent structural unit AF may be a structural unit described below as a structural unit L2 that has an aforementioned monovalent structural unit having an N-aryl phenoxazine skeleton as a substituent R.

### (Trivalent or Higher Structural Unit AF)

A trivalent or higher structural unit AF has an N-aryl phenoxazine skeleton, and has three or more bonding sites with other structural units. In one embodiment, the trivalent or higher structural unit AF preferably has a structure in which three or more hydrogen atoms have been removed from an N-aryl phenoxazine skeleton. This embodiment includes structures in which a hydrogen atom has been removed from a substituent on the N-aryl phenoxazine skeleton.

The trivalent or higher structural unit AF is preferably not higher than hexavalent. In one embodiment, a trivalent or tetravalent structural unit AF is preferred. In one embodiment, the charge transport polymer preferably includes a structural unit shown below as a trivalent or higher structural unit B1 having charge transport properties. However, the trivalent or tetravalent structural unit AF is not limited to the following structural units.

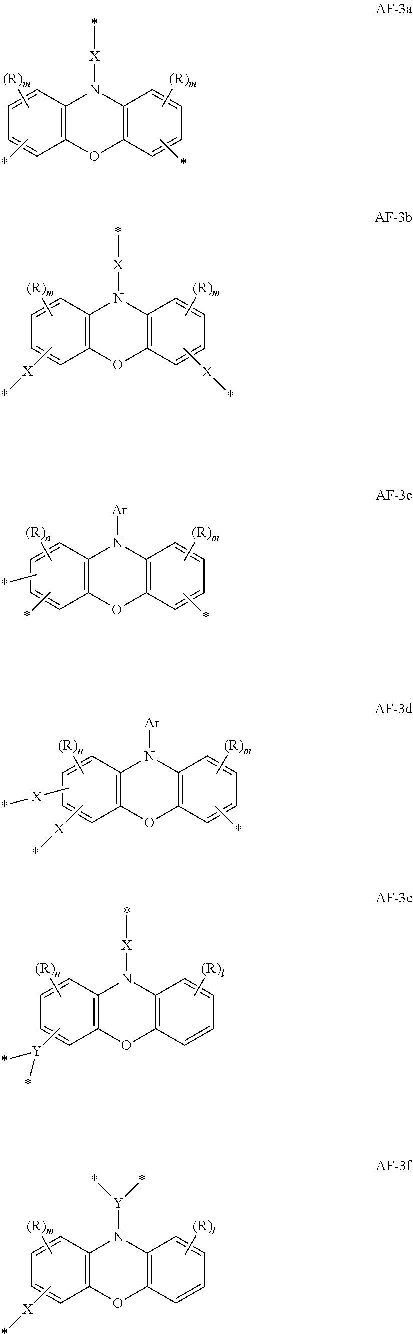

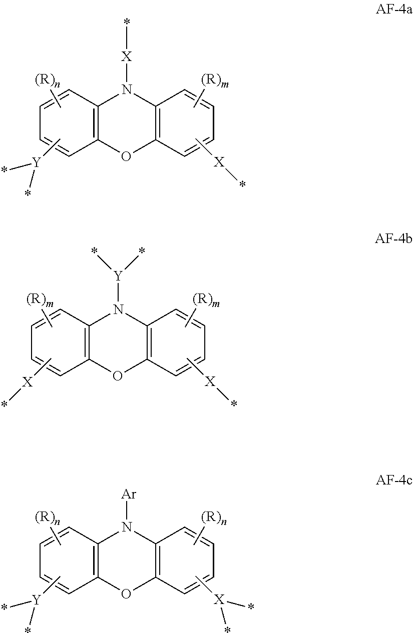

In the above structural units, 1 represents an integer of 0 to 4, m represents an integer of 0 to 3 and n represents an integer of 0 to 2, with each representing a number of R groups. The symbol “*” represents a bonding site with another structural unit. R, Ar, X and Y are the same as described above in relation to the monovalent structural unit AF and the divalent structural unit AF.

Specific examples of preferred trivalent and tetravalent structural units AF are shown below. However, the trivalent or tetravalent structural unit AF is not limited to the following structural units.

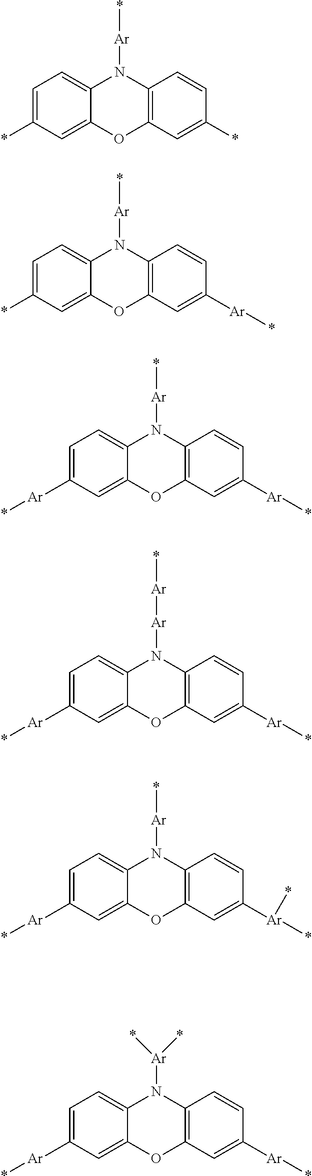

In the formulas, each Ar represents a substituted or unsubstituted arylene group or arenetriyl group of 6 to 30 carbon atoms. The symbol “*” represents a bonding site with another structural unit.

Specific examples of even more preferred trivalent and tetravalent structural units AF are shown below. The symbol “*” represents a bonding site with another structural unit.

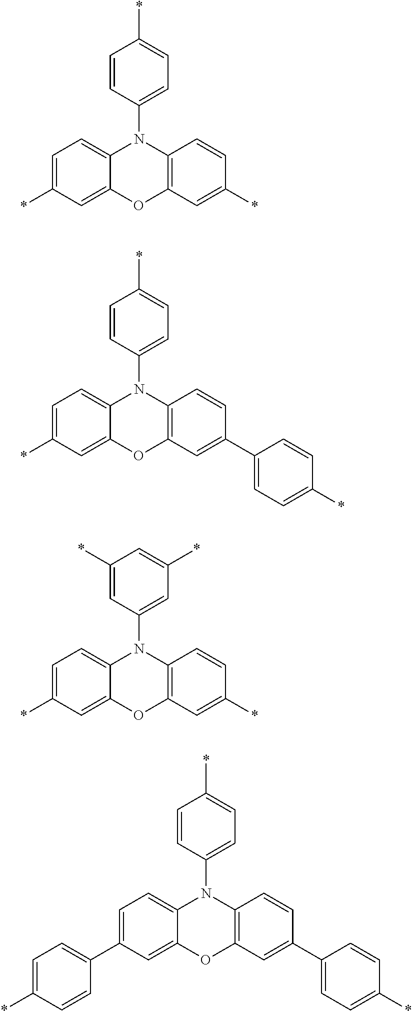

In another embodiment, the trivalent or tetravalent structural unit AF may be a structural unit described below as a structural unit B2 that has an aforementioned monovalent structural unit having an N-aryl phenoxazine skeleton as a substituent.

In one embodiment, the charge transport polymer preferably contains at least one structural unit selected from the group consisting of the divalent structural unit AF and the trivalent structural unit AF. Although not a particular limitation, in this embodiment, preferred examples of the divalent and trivalent structural units AF include those shown below.

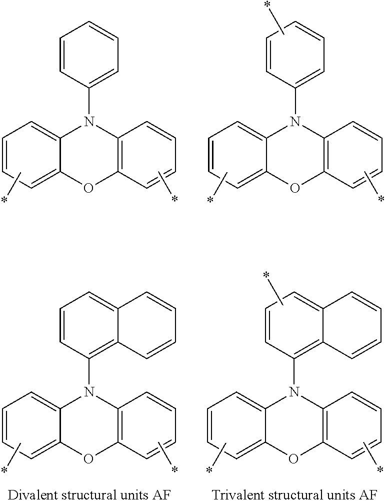

In one embodiment, in addition to having at least one monovalent or higher structural unit AF described above (hereafter also referred to as the structural unit L1, the structural unit T1 and the structural unit B1), the charge transport polymer may also contain another monovalent or higher structural unit having charge transport properties that is different from these structural units AF. This optionally included structural unit is preferably a structural unit that is not higher than hexavalent, and is more preferably tetravalent or lower. In one embodiment, the charge transport polymer may also contain at least one structural unit selected from among divalent structural units L2, monovalent structural units T2 and trivalent or higher structural units B2 described below.

### (Structural Unit L2)

The structural unit L2 is a divalent structural unit having charge transport properties. There are no particular limitations on the structural unit L2, provided it includes an atom grouping that has the ability to transport an electric charge. For example, the structural unit L2 may be selected from among substituted or unsubstituted structures including aromatic amine structures, carbazole structures, thiophene structures, bithiophene structures, fluorene structures, benzene structures, biphenyl structures, terphenyl structures, naphthalene structures, anthracene structures, tetracene structures, phenanthrene structures, dihydrophenanthrene structures, pyridine structures, pyrazine structures, quinoline structures, isoquinoline structures, quinoxaline structures, acridine structures, diazaphenanthrene structures, furan structures, pyrrole structures, oxazole structures, oxadiazole structures, thiazole structures, thiadiazole structures, triazole structures, benzothiophene structures, benzoxazole structures, benzoxadiazole structures, benzothiazole structures, benzothiadiazole structures, benzotriazole structures, and structures containing one, or two or more, of the above structures.

In one embodiment, from the viewpoint of obtaining superior hole transport properties, the structural unit L2 is preferably selected from among substituted or unsubstituted structures including aromatic amine structures, carbazole structures, thiophene structures, fluorene structures, benzene structures, pyrrole structures, and structures containing one, or two or more, of these structures. In one embodiment, the structural unit L2 is more preferably selected from among substituted or unsubstituted structures including aromatic amine structures, carbazole structures, and structures containing one, or two or more, of these structures. In another embodiment, from the viewpoint of obtaining superior electron transport properties, the structural unit L2 is preferably selected from among substituted or unsubstituted structures including fluorene structures, benzene structures, phenanthrene structures, pyridine structures, quinoline structures, and structures containing one, or two or more, of these structures. Specific examples of the structural unit L2 are shown below.

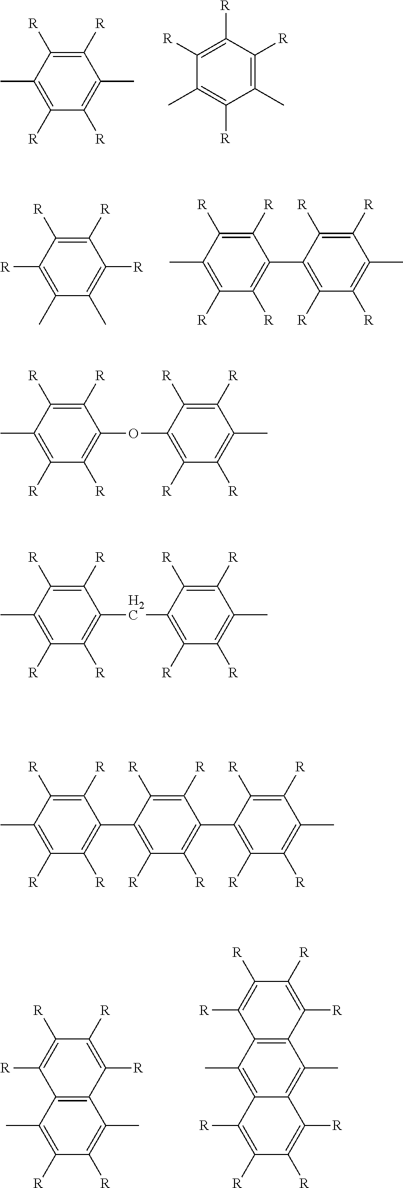

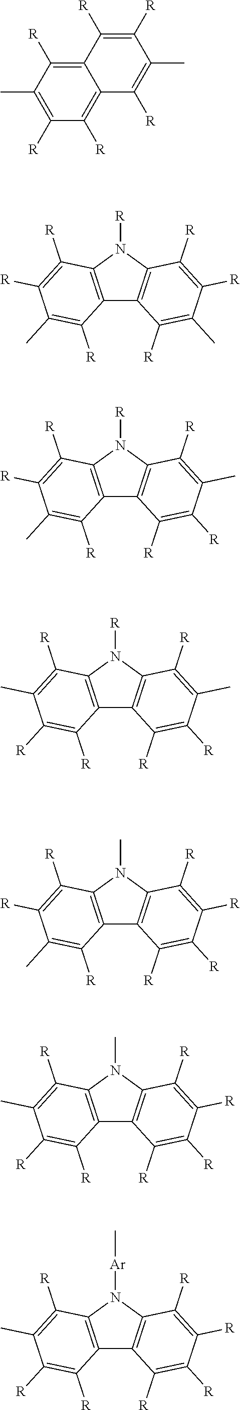

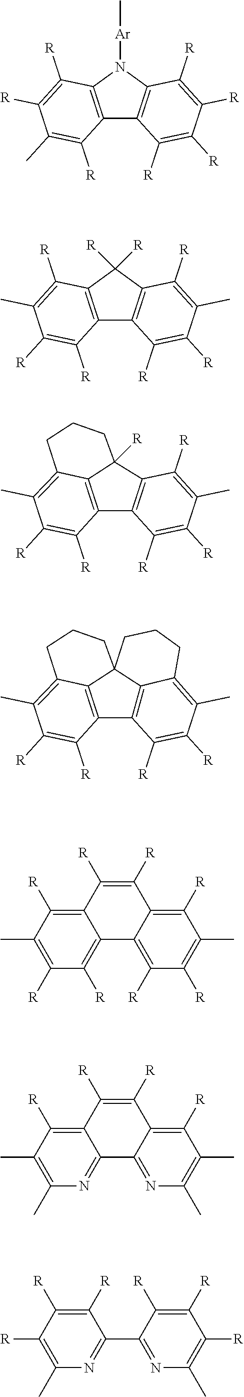

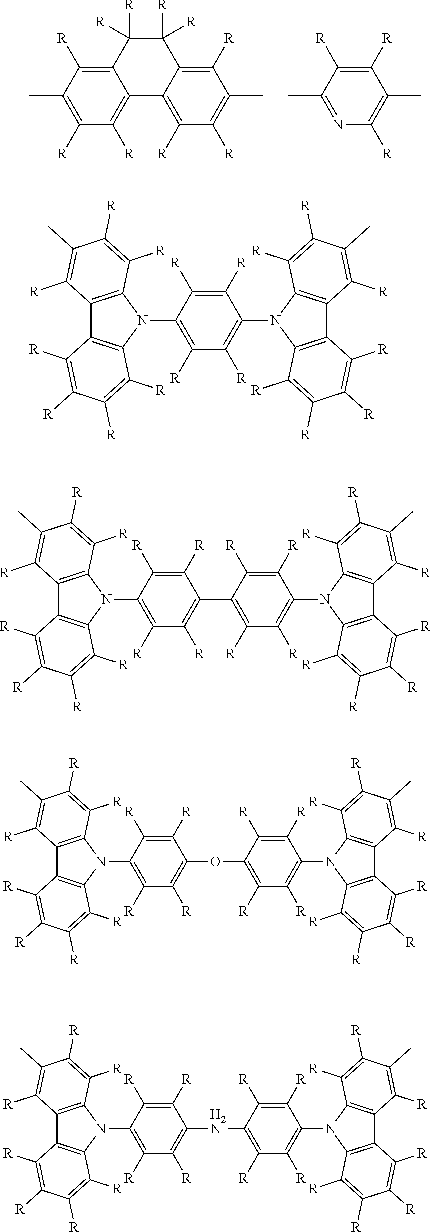

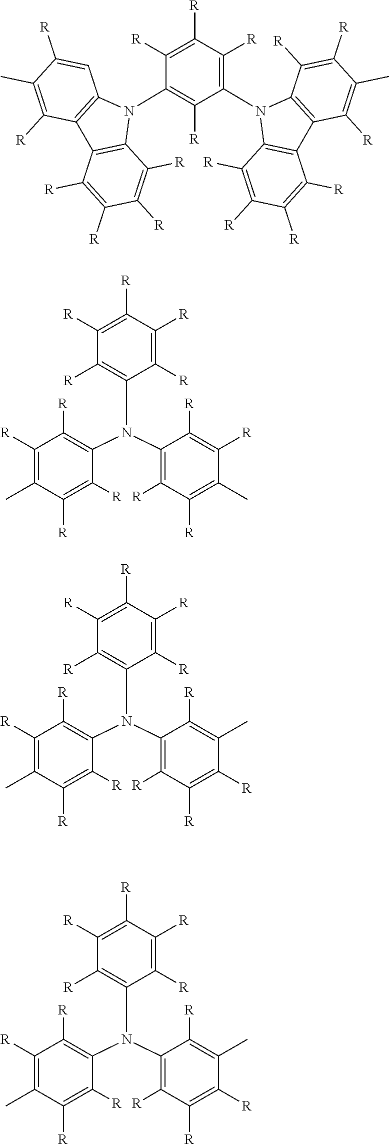

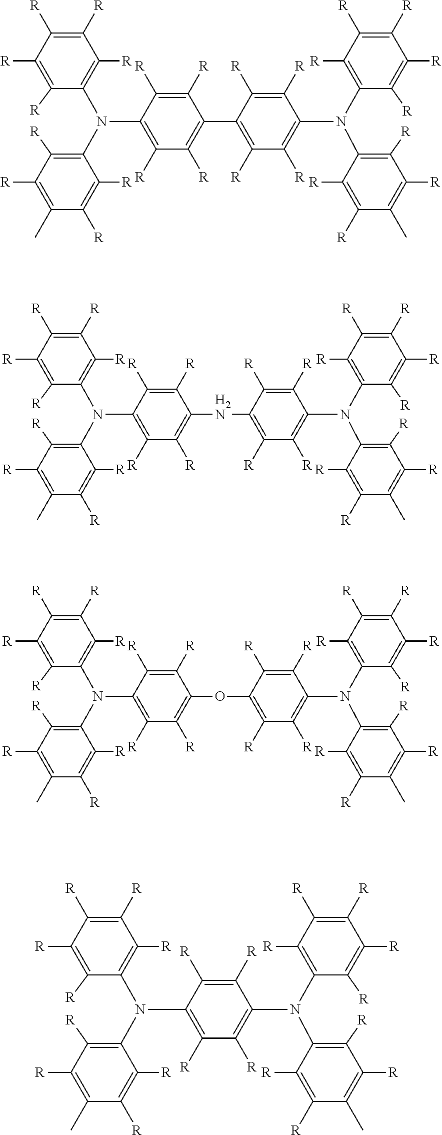

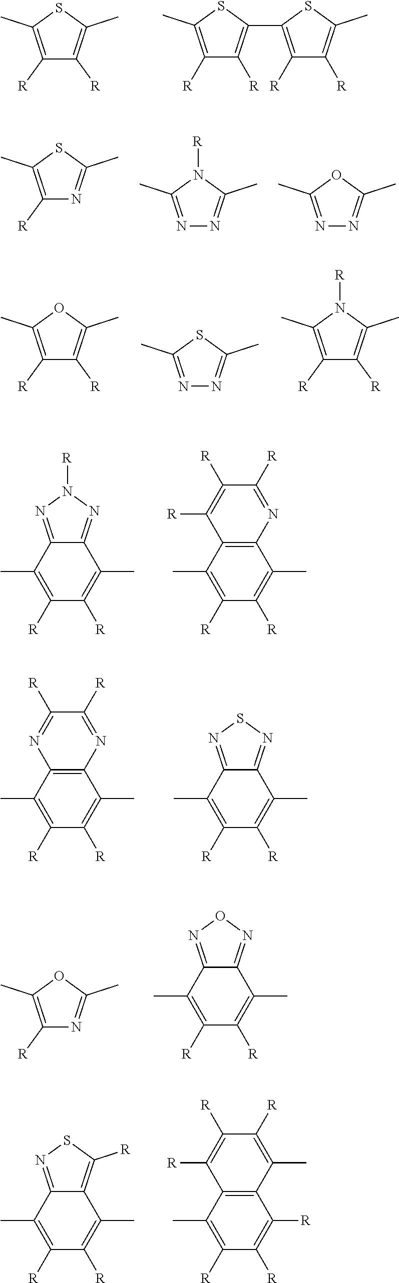

Each R independently represents a hydrogen atom or a substituent. It is preferable that each R is independently selected from a group consisting of —R¹, —OR², —SR³, —OCOR⁴, —COOR⁵, —SiR⁶R⁷R⁸, halogen atoms, and groups containing a polymerizable functional group described below. Each of R¹ to R⁸ independently represents a hydrogen atom, a linear, cyclic or branched alkyl group of 1 to 22 carbon atoms, or an aryl group or heteroaryl group of 2 to 30 carbon atoms. An aryl group is an atom grouping in which one hydrogen atom has been removed from an aromatic hydrocarbon. A heteroaryl group is an atom grouping in which one hydrogen atom has been removed from an aromatic heterocycle. However, in embodiments of the present invention, this heteroaryl group excludes groups having an N-aryl phenoxazine skeleton. The alkyl group may be further substituted with an aryl group or heteroaryl group of 2 to 20 carbon atoms, and the aryl group or heteroaryl group may be further substituted with a linear, cyclic or branched alkyl group of 1 to 22 carbon atoms. R is preferably a hydrogen atom, an alkyl group, an aryl group, or an alkyl-substituted aryl group. Ar represents an arylene group or heteroarylene group of 2 to 30 carbon atoms. An arylene group is an atom grouping in which two hydrogen atoms have been removed from an aromatic hydrocarbon. A heteroarylene group is an atom grouping in which two hydrogen atoms have been removed from an aromatic heterocycle. However, in embodiments of the present invention, the heteroaryl group or heteroarylene group excludes groups having an N-aryl phenoxazine skeleton. Ar is preferably an arylene group, and is more preferably a phenylene group.

Examples of the aromatic hydrocarbon include monocyclic hydrocarbons, condensed ring hydrocarbons, and polycyclic hydrocarbons in which two or more hydrocarbons selected from among monocyclic hydrocarbons and condensed ring hydrocarbons are bonded together via single bonds. Examples of the aromatic heterocycles include monocyclic heterocycles, condensed ring heterocycles, and polycyclic heterocycles in which two or more heterocycles selected from among monocyclic heterocycles and condensed ring heterocycles are bonded together via single bonds.

### (Structural Unit B2)

The structural unit B2 is a trivalent or higher structural unit that constitutes a branched portion in those cases where the charge transport polymer has a branched structure. From the viewpoint of improving the durability of the organic electronic element, the structural unit B2 is preferably not higher than hexavalent, and is more preferably either trivalent or tetravalent. The structural unit B2 is preferably a unit that has charge transport properties. For example, from the viewpoint of improving the durability of the organic electronic element, the structural unit B2 is preferably selected from among substituted or unsubstituted structures including triphenylamine structures, carbazole structures, condensed polycyclic aromatic hydrocarbon structures, and structures containing one, or two or more, of these structures. Specific examples of the structural unit B2 are shown below.

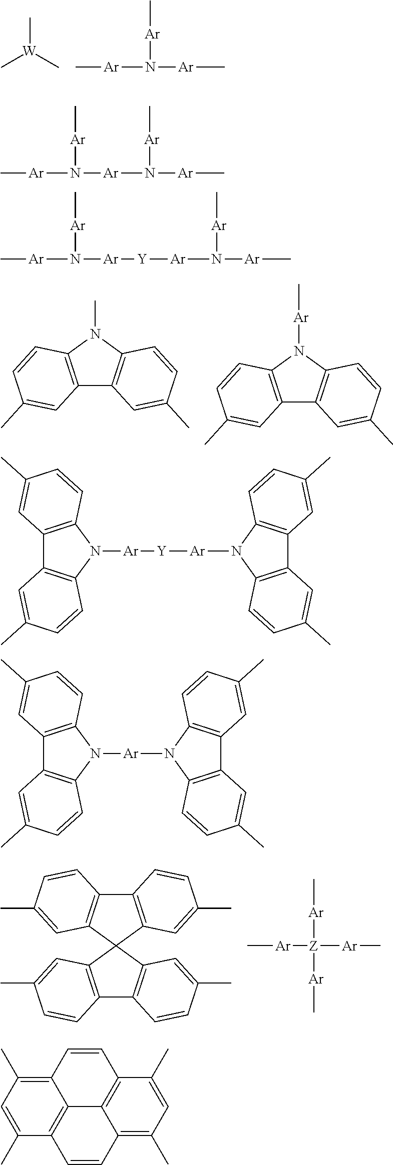

W represents a trivalent linking group, and for example, represents an arenetriyl group or heteroarenetriyl group of 2 to 30 carbon atoms. An arenetriyl group is an atom grouping in which three hydrogen atoms have been removed from an aromatic hydrocarbon. A heteroarenetriyl group is an atom grouping in which three hydrogen atoms have been removed from an aromatic heterocycle. Each Ar independently represents a divalent linking group, and for example, may represent an arylene group or heteroarylene group of 2 to 30 carbon atoms. Here, the above heteroarenetriyl group and heteroarylene group exclude groups having an N-aryl phenoxazine skeleton. Ar preferably represents an arylene group, and more preferably a phenylene group. Y represents a divalent linking group, and examples include divalent groups in which an additional hydrogen atom has been removed from any of the R groups having one or more hydrogen atoms (but excluding groups containing a polymerizable functional group) described in relation to the structural unit L2. Z represents a carbon atom, a silicon atom or a phosphorus atom. In the structural units, the benzene rings and Ar groups may have a substituent, and examples of the substituent include the R groups in the structural unit L2.

### (Structural Unit T2)

In the charge transport polymer, the structural unit T2 is a monovalent structural unit that constitutes a terminal portion of the charge transport polymer. There are no particular limitations on the structural unit T2, which may be selected from among substituted or unsubstituted structures including aromatic hydrocarbon structures, aromatic heterocyclic structures, and structures containing one, or two or more, of these structures. In one embodiment, from the viewpoint of imparting durability to the charge transport polymer without impairing the charge transport properties, the structural unit T2 is preferably a substituted or unsubstituted aromatic hydrocarbon structure, and is more preferably a substituted or unsubstituted benzene structure. Further, in another embodiment, when the charge transport polymer has a polymerizable functional group at a terminal portion in the manner described below, the structural unit T2 may be a polymerizable structure (for example, a polymerizable functional group such as a pyrrolyl group).

Specific examples of the structural unit T2 are shown below.

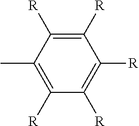

R is the same as R described in relation to the structural unit L2. In those cases where the charge transport polymer has a polymerizable functional group at a terminal portion, it is preferable that at least one R is a group containing a polymerizable functional group.

### (Group Containing a Polymerizable Functional Group)

In one embodiment, from the viewpoint of enabling the polymer to be cured by a polymerization reaction, thereby changing the solubility in solvents, the charge transport polymer preferably has at least one group containing a polymerizable functional group. This “polymerizable functional group” refers to a group which is able to form bonds upon the application of heat and/or light.

Examples of the polymerizable functional group include groups having a carbon-carbon multiple bond (such as a vinyl group, allyl group, butenyl group, ethynyl group, acryloyl group, acryloyloxy group, acryloylamino group, methacryloyl group, methacryloyloxy group, methacryloylamino group, vinyloxy group and vinylamino group), groups having a small ring (including cycloalkyl groups such as a cyclopropyl group and cyclobutyl group; cyclic ether groups such as an epoxy group (oxiranyl group) and oxetane group (oxetanyl group); diketene groups; episulfide groups; lactone groups; and lactam groups); and heterocyclic groups (such as a furanyl group, pyrrolyl group, thiophenyl group and siloyl group). Particularly preferred polymerizable functional groups include a vinyl group, acryloyl group, methacryloyl group, epoxy group and oxetane group, and from the viewpoints of improving the reactivity and the characteristics of the organic electronic element, a vinyl group, oxetane group or epoxy group is even more preferred.

From the viewpoints of increasing the degree of freedom associated with the polymerizable functional group and facilitating the polymerization reaction, the main skeleton of the charge transport polymer and the polymerizable functional group are preferably linked via an alkylene chain.

Further, in the case where, for example, the organic layer is to be formed on an electrode, from the viewpoint of enhancing the affinity with hydrophilic electrodes of ITO or the like, the main backbone and the polymerizable functional group are preferably linked via a hydrophilic chain such as an ethylene glycol chain or a diethylene glycol chain. Moreover, from the viewpoint of simplifying preparation of the monomer used for introducing the polymerizable functional group, the charge transport polymer may have an ether linkage or an ester linkage at the terminal of the alkylene chain and/or the hydrophilic chain, namely, at the linkage site between these chains and the polymerizable functional group, and/or at the linkage site between these chains and the charge transport polymer backbone. The aforementioned “group containing a polymerizable functional group” means a polymerizable functional group itself, or a group composed of a combination of a polymerizable functional group and an alkylene chain or the like. Examples of groups that can be used favorably as this group containing a polymerizable functional group include the groups exemplified in WO 2010/140553.

The polymerizable functional group may be introduced at a terminal portion of the charge transport polymer (namely, a structural unit T), at a portion other than a terminal portion (namely, a structural unit L or B), or at both a terminal portion and a portion other than a terminal. From the viewpoint of the curability, the polymerizable functional group is preferably introduced at least at a terminal portion, and from the viewpoint of achieving a combination of favorable curability and charge transport properties, is preferably introduced only at terminal portions. Further, in those cases where the charge transport polymer has a branched structure, the polymerizable functional group may be introduced within the main chain of the charge transport polymer, within a side chain, or within both the main chain and a side chain.

From the viewpoint of contributing to a change in the solubility, the polymerizable functional group is preferably included in the charge transport polymer in a large amount. On the other hand, from the viewpoint of not impeding the charge transport properties, the amount included in the charge transport polymer is preferably kept small. The amount of the polymerizable functional group may be set as appropriate with due consideration of these factors.

For example, from the viewpoint of obtaining a satisfactory change in the solubility, the number of polymerizable functional groups per one molecule of the charge transport polymer is preferably at least 2, and more preferably 3 or greater. Further, from the viewpoint of maintaining good charge transport properties, the number of polymerizable functional groups is preferably not more than 1,000, and more preferably 500 or fewer.

The number of polymerizable functional groups per one molecule of the charge transport polymer can be determined as an average value from the amount of the polymerizable functional group used in synthesizing the charge transport polymer (for example, the amount added of the monomer having the polymerizable functional group), the amounts added of the monomers corresponding with the various structural units, and the weight average molecular weight of the charge transport polymer and the like. Further, the number of polymerizable functional groups can also be calculated as an average value using the ratio between the integral of the signal attributable to the polymerizable functional group and the integral of the total spectrum in the ¹H-NMR (nuclear magnetic resonance) spectrum of the charge transport polymer, and the weight average molecular weight of the charge transport polymer and the like. In terms of ease of calculation, if the amounts added of the various components are clear, then the number of polymerizable functional groups is preferably determined from these amounts.

### (Partial Structures of Charge Transport Polymer)

Examples of partial structures contained in the charge transport polymer are described below. However, the charge transport polymer is not limited to polymers having the following partial structures. In the partial structures, “L” represents a divalent structural unit having charge transport properties, “T” represents a monovalent structural unit that constitutes a terminal group, and “B” represents a trivalent or tetravalent structural unit that constitutes a branched structure. The symbol “*” represents a bonding site with another structural unit. In the following partial structures, the plurality of L units may be the same structural units or mutually different structural units. This also applies for the T and B units.

### Linear Charge Transport Polymer

T-L-L-L-L-L-*

Charge Transport Polymers having Branched Structures

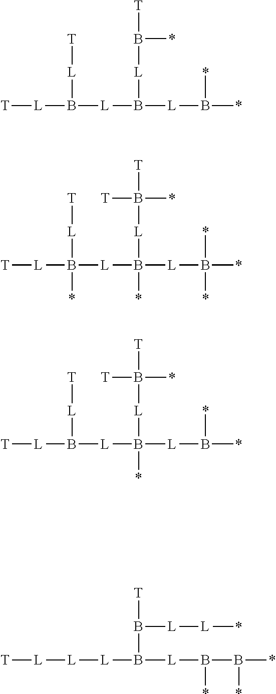

In the above partial structures, the structural unit L represents L1 and/or L2, whereas T represents T1 and/or T2, and B represents B1 and/or B2. In one embodiment, the charge transport polymer contains at least one structural unit selected from among structural units L1, T1 and B1 as the structural unit AF having an N-aryl phenoxazine skeleton, and may also contain an optional combination of other structural units L2, T2 and B2.

In one embodiment, the charge transport polymer preferably contains at least one structural unit selected from the group consisting of divalent structural units L1 having an N-aryl phenoxazine skeleton and trivalent or higher structural units B1 having an N-aryl phenoxazine skeleton. The charge transport polymer preferably contains at least a trivalent or higher structural unit B1 having an N-aryl phenoxazine skeleton.

In one embodiment, the charge transport polymer contains at least one structural unit selected from the group consisting of the divalent structural units L1 and the trivalent or higher structural units B1 as the structural unit AF having an N-aryl phenoxazine skeleton, and may also contain at least one structural unit selected from the group consisting of divalent structural units L2 and trivalent or higher structural units B2, which have charge transport properties but differ from the aforementioned structural unit AF.

In one embodiment, the charge transport polymer preferably also contains, in addition to the structural unit AF having an N-aryl phenoxazine skeleton, an aforementioned divalent structural unit L2 having charge transport properties. Here, the divalent structural unit L2 is preferably at least one structure selected from the group consisting of aromatic amine structures, carbazole structures, thiophene structures, benzene structures and fluorene structures. The benzene structures preferably include a p-phenylene structure or an m-phenylene structure. The divalent structural unit L2 more preferably contains an aromatic amine structure and/or a carbazole structure. The aromatic amine structure may be an aniline structure, but is preferably a triarylamine structure, and more preferably a triphenylamine structure.

In one embodiment, the charge transport polymer preferably contains at least one of the trivalent or higher structural units B1 and B2, thus having a structure that is branched in three or more directions. In this type of embodiment, the charge transport polymer may either contain a trivalent or higher structural unit B1, or may contain the structural unit B2, with the N-aryl phenoxazine skeleton introduced into the polymer by also including the structural unit L1 and/or T1.

In this description, a “structure that is branched in three or more directions” means that among the various chains within a single molecule of the charge transport polymer, if the chain that has the highest degree of polymerization is deemed the main chain, then one or more side chains having a degree of polymerization that is either the same as, or smaller than, that of the main chain also exist in the molecule. The “degree of polymerization” represents the number of monomer units used in synthesizing the charge transport polymer that are contained within one molecule of the charge transport polymer. Further, in this description, a “side chain” means a chain that is different from the main chain of the charge transport polymer and has at least one structural unit, whereas other moieties outside of this definition are deemed substituents.

In another embodiment, the charge transport polymer may contain a structure having an N-aryl phenoxazine skeleton as a substituent in an aforementioned structural unit L, T or B. For example, the charge transport polymer may contain a monovalent structural unit T1 having an N-aryl phenoxazine skeleton as the substituent R in one of the structures exemplified above as the structural unit L2.

### (Proportion of Structural Unit AF)

In embodiments of the present invention, by including a structure having an N-aryl phenoxazine skeleton within the charge transport polymer, improvements in the performance of the polymer including the durability and the emission lifespan can be achieved with ease. In one embodiment, from the viewpoint of obtaining superior durability, the proportion of the structural unit AF in the charge transport polymer, relative to the total of all the structural units, is preferably at least 1 mol %, more preferably at least 3 mol %, and most preferably 5 mol % or greater.

On the other hand, from the viewpoint of further enhancing the charge transport properties of the charge transport polymer, the charge transport polymer preferably also contains one or more other structural units having charge transport properties besides the structural unit AF. From this type of viewpoint, in one embodiment, the proportion of the structural unit AF, relative to the total of all the structural units, is preferably not more than 90 mol %, more preferably not more than 80 mol %, and even more preferably 70 mol % or less.

Accordingly, in one embodiment, the proportion of the structural unit AF having an N-aryl phenoxazine skeleton in the charge transport polymer, relative to the total of all the structural units, is preferably within a range from 1 to 90 mol %, more preferably from 3 to 80 mol %, and even more preferably from 5 to 70 mol %. The above proportion of the structural unit AF is also preferred in terms of obtaining a charge transport polymer having a molecular weight that is suitable for a charge transport material. Here, the proportion of the structural unit AF means the total amount of the one or more structural units L1, T1 and B1 that constitute the polymer.

### (Proportions of Structural Units L, T and B)

In the charge transport polymer, from the viewpoint of achieving satisfactory charge transport properties, the proportion of the divalent structural unit L, relative to the total of all the structural units, is preferably at least 10 mol %, more preferably at least 20 mol %, and even more preferably 30 mol % or higher. If the structural unit T and the optionally included structural unit B are taken into consideration, then the proportion of the structural unit L is preferably not more than 95 mol %, more preferably not more than 90 mol %, and even more preferably 85 mol % or less.

Here, the structural unit L means an arbitrary combination of the structural unit L1 and other structural units L2. In one embodiment, from the viewpoint of ensuring satisfactory manifestation of the effects of the structural unit AF having an N-aryl phenoxazine skeleton, the proportion of the structural unit L1 relative to the combined total of L1 and L2 is preferably at least 1 mol %, more preferably at least 3 mol %, and even more preferably 5 mol % or greater.

From the viewpoint of improving the characteristics of the organic electronic element, or from the viewpoint of suppressing any increase in viscosity and enabling the synthesis of the charge transport polymer to be performed favorably, the proportion of the structural unit T within the charge transport polymer, relative to the total of all the structural units, is preferably at least 5 mol %, more preferably at least 10 mol %, and even more preferably 15 mol % or greater. Further, from the viewpoint of ensuring satisfactory charge transport properties, the proportion of the structural unit T is preferably not more than 60 mol %, more preferably not more than 55 mol %, and even more preferably 50 mol % or less.

Here, the structural unit T means an arbitrary combination of the structural unit T1 and other structural units T2. In one embodiment, from the viewpoint of ensuring satisfactory manifestation of the effects of the structural unit AF having an N-aryl phenoxazine skeleton, the proportion of the structural unit T1 relative to the combined total of T1 and T2 is preferably at least 1 mol %, more preferably at least 3 mol %, and even more preferably 5 mol % or greater.

In those cases where the charge transport polymer includes a trivalent or higher structural unit B, from the viewpoint of improving the durability of the organic electronic element, the proportion of the structural unit B, relative to the total of all the structural units, is preferably at least 1 mol %, more preferably at least 5 mol %, and even more preferably 10 mol % or higher. Further, from the viewpoints of suppressing any increase in viscosity and enabling the synthesis of the charge transport polymer to be performed favorably, or from the viewpoint of ensuring satisfactory charge transport properties, the proportion of the structural unit B is preferably not more than 50 mol %, more preferably not more than 40 mol %, and even more preferably 30 mol % or less.

Here, the structural unit B means an arbitrary combination of the structural unit B1 and other structural units B2. In one embodiment, from the viewpoint of ensuring satisfactory manifestation of the effects of the structural unit AF having an N-aryl phenoxazine skeleton, the proportion of the structural unit B1 relative to the combined total of B1 and B2 is preferably at least 1 mol %, more preferably at least 3 mol %, and even more preferably 5 mol % or greater.

In those cases where the charge transport polymer has a polymerizable functional group, from the viewpoint of ensuring efficient curing of the charge transport polymer, the proportion of the polymerizable functional group, relative to the total of all the structural units, is preferably at least 0.1 mol %, more preferably at least 1 mol %, and even more preferably 3 mol % or higher. Further, from the viewpoint of ensuring favorable charge transport properties, the proportion of the polymerizable functional group is preferably not more than 70 mol %, more preferably not more than 60 mol %, and even more preferably 50 mol % or less. Here, the “proportion of the polymerizable functional group” refers to the proportion of structural units having the polymerizable functional group.

Considering the balance between the charge transport properties, the durability, and the productivity and the like, the ratio (molar ratio) between the structural unit L and the structural unit T is preferably L:T=100:(1 to 70), more preferably 100:(3 to 50), and even more preferably 100:(5 to 30). Further, in those cases where the charge transport polymer also includes the structural unit B, the ratio (molar ratio) between the structural unit L, the structural unit T and the structural unit B is preferably L:T:B=100:(10 to 200):(10 to 100), more preferably 100:(20 to 180):(20 to 90), and even more preferably 100:(40 to 160):(30 to 80).

The aforementioned structural unit L means an arbitrary combination of the structural unit L1 having an N-aryl phenoxazine skeleton and other divalent structural units L2. Further, the aforementioned structural unit B means an arbitrary combination of the structural unit B1 having an N-aryl phenoxazine skeleton and other trivalent or higher structural units B2. Moreover, the aforementioned structural unit T means an arbitrary combination of the structural unit T1 having an N-aryl phenoxazine skeleton and other monovalent structural units T2. Here, the ratio between the structural units L1 and L2, the ratio between the structural units T1 and T2, and the ratio between the structural units B1 and B2 are as described above, and in one embodiment, the charge transport polymer is presumed to contain at least one of the structural units L1, B1 and T1.

The proportion of each structural unit can be determined from the amount added of the monomer corresponding with that structural unit during synthesis of the charge transport polymer. Further, the proportion of each structural unit can also be calculated using the integral of the spectrum attributable to the structural unit in the ¹H-NMR spectrum of the charge transport polymer, and the weight average molecular weight or the like of the structural unit. In terms of convenience, if the amounts added of each monomer are clear, then the proportion of each structural unit preferably employs the value determined using the amount added of the monomer.

### (Number Average Molecular Weight)

The number average molecular weight of the charge transport polymer can be adjusted appropriately with due consideration of the solubility in solvents and the film formability and the like. From the viewpoint of ensuring superior charge transport properties, the number average molecular weight is preferably at least 500, more preferably at least 1,000, and even more preferably 2,000 or greater. Further, from the viewpoints of maintaining favorable solubility in solvents and facilitating the preparation of ink compositions, the number average molecular weight is preferably not more than 1,000,000, more preferably not more than 100,000, and even more preferably 50,000 or less.

### (Weight Average Molecular Weight)

The weight average molecular weight of the charge transport polymer can be adjusted appropriately with due consideration of the solubility in solvents and the film formability and the like. From the viewpoint of ensuring superior charge transport properties, the weight average molecular weight is preferably at least 1,000, more preferably at least 5,000, and even more preferably 10,000 or greater. Further, from the viewpoints of maintaining favorable solubility in solvents and facilitating the preparation of ink compositions, the weight average molecular weight is preferably not more than 1,000,000, more preferably not more than 700,000, and even more preferably 400,000 or less.

The number average molecular weight and the weight average molecular weight can be measured by gel permeation chromatography (GPC), using a calibration curve of standard polystyrenes.

### (Production Method)

The charge transport polymer can be produced by various synthesis methods, and there are no particular limitations. For example, conventional coupling reactions such as the Suzuki coupling, Negishi coupling, Sonogashira coupling, Stille coupling and Buchwald-Hartwig coupling reactions can be used. The Suzuki coupling is a reaction in which a cross-coupling reaction is initiated between an aromatic boronic acid derivative and an aromatic halide using a Pd catalyst. By using a Suzuki coupling, the charge transport polymer can be produced easily by bonding together the desired aromatic rings.

In the coupling reaction, a Pd(0) compound, Pd(II) compound, or Ni compound or the like is used as a catalyst. Further, a catalyst species generated by mixing a precursor such as tris(dibenzylideneacetone)dipalladium(0) or palladium(II) acetate with a phosphine ligand can also be used. Reference may also be made to WO 2010/140553 in relation to synthesis methods for the charge transport polymer.

### [Dopant]

In those cases where the charge transport material is used to form an organic electronic element, the charge transport material may also contain known additives for organic electronic materials. In one embodiment, the charge transport material may also contain a dopant. There are no particular limitations on the dopant, provided it is a substance that yields a doping effect upon addition to the charge transport material, enabling an improvement in the charge transport properties. Doping includes both p-type doping and n-type doping. In p-type doping, a substance that functions as an electron acceptor is used as the dopant, whereas in n-type doping, a substance that functions as an electron donor is used as the dopant. To improve the hole transport properties, p-type doping is preferably used, whereas to improve the electron transport properties, n-type doping is preferably used. The dopant used in the charge transport material may be a dopant that exhibits either a p-type doping effect or an n-type doping effect. Further, a single type of dopant may be added alone, or a mixture of a plurality of dopant types may be added.

The dopants used in p-type doping are electron-accepting compounds, and examples include Lewis acids, protonic acids, transition metal compounds, ionic compounds, halogen compounds and π-conjugated compounds. Specific examples include Lewis acids such as FeCl₃, PF₅, AsF₅, SbF₅, BF₅, BCl₃ and BBr₃; protonic acids, including inorganic acids such as HF, HCl, HBr, HNO₃, H₂SO₄ and HClO₄, and organic acids such as benzenesulfonic acid, p-toluenesulfonic acid, dodecylbenzenesulfonic acid, polyvinylsulfonic acid, methanesulfonic acid, trifluoromethanesulfonic acid, trifluoroacetic acid, 1-butanesulfonic acid, vinylphenylsulfonic acid and camphorsulfonic acid; transition metal compounds such as FeOCl, TiCl₄, ZrCl₄, HfCl₄, NbF₅, AlCl₃, NbCl₅, TaCl₅ and MoF₅; ionic compounds, including salts containing a perfluoro anion such as a tetrakis(pentafluorophenyl)borate ion, tris(trifluoromethanesulfonyl)methide ion, bis(trifluoromethanesulfonyl)imide ion, hexafluoroantimonate ion, AsF₆⁻ (hexafluoroarsenate ion), BF₄⁻ (tetrafluoroborate ion) or PF₆⁻ (hexafluorophosphate ion), and salts having a conjugate base of an aforementioned protonic acid as an anion; halogen compounds such as Cl₂, Br₂, I₂, ICl, ICl₃, IBr and IF; and π-conjugated compounds such as TCNE (tetracyanoethylene) and TCNQ (tetracyanoquinodimethane). Further, the electron-accepting compounds disclosed in JP 2000-36390 A, JP 2005-75948 A, and JP 2003-213002 A and the like can also be used. Lewis acids, ionic compounds, and π-conjugated compounds and the like are preferred.

The dopants used in n-type doping are electron-donating compounds, and examples include alkali metals such as Li and Cs; alkaline earth metals such as Mg and Ca; salts of alkali metals and/or alkaline earth metals such as LiF and Cs₂CO₃; metal complexes; and electron-donating organic compounds.

In those cases where the charge transport polymer has a polymerizable functional group, in order to make it easier to change the solubility of the organic layer, a compound that can function as a polymerization initiator for the polymerizable functional group is preferably used as the dopant.

### [Other Optional Components]

The charge transport material may also contain charge transport low-molecular weight compounds, or other polymers or the like.

### [Contents]

From the viewpoint of obtaining favorable charge transport properties, the amount of the charge transport polymer, relative to the total mass of the organic electronic material, is preferably at least 50% by mass, more preferably at least 70% by mass, and even more preferably 80% by mass or greater. This amount may be 100% by mass.

When a dopant is included, from the viewpoint of improving the charge transport properties of the charge transport material, the amount of the dopant relative to the total mass of the charge transport material is preferably at least 0.01% by mass, more preferably at least 0.1% by mass, and even more preferably 0.5% by mass or greater. Further, from the viewpoint of maintaining favorable film formability, the amount of the dopant relative to the total mass of the charge transport material is preferably not more than 50% by mass, more preferably not more than 30% by mass, and even more preferably 20% by mass or less.

### <Ink Composition>

In one embodiment, an ink composition contains the charge transport material of an embodiment described above and a solvent that is capable of dissolving or dispersing that material. By using this ink composition, an organic layer can be formed easily using a simple coating method.

### [Solvent]

Water, organic solvents, or mixed solvents thereof can be used as the solvent. Examples of the organic solvent include alcohols such as methanol, ethanol and isopropyl alcohol; alkanes such as pentane, hexane and octane; cyclic alkanes such as cyclohexane; aromatic hydrocarbons such as benzene, toluene, xylene, mesitylene, tetralin and diphenylmethane; aliphatic ethers such as ethylene glycol dimethyl ether, ethylene glycol diethyl ether and propylene glycol-1-monomethyl ether acetate; aromatic ethers such as 1,2-dimethoxybenzene, 1,3-dimethoxybenzene, anisole, phenetole, 2-methoxytoluene, 3-methoxytoluene, 4-methoxytoluene, 2,3-dimethylanisole and 2,4-dimethylanisole; aliphatic esters such as ethyl acetate, n-butyl acetate, ethyl lactate and n-butyl lactate; aromatic esters such as phenyl acetate, phenyl propionate, methyl benzoate, ethyl benzoate, propyl benzoate and n-butyl benzoate; amide-based solvents such as N,N-dimethylformamide and N,N-dimethylacetamide; as well as dimethyl sulfoxide, tetrahydrofuran, acetone, chloroform and methylene chloride and the like. Preferred solvents include aromatic hydrocarbons, aliphatic esters, aromatic esters, aliphatic ethers, and aromatic ethers and the like.

### [Polymerization Initiator]

In those cases where the charge transport polymer has a polymerizable functional group, the ink composition preferably contains a polymerization initiator. Conventional radical polymerization initiators, cationic polymerization initiators, and anionic polymerization initiators and the like can be used as the polymerization initiator. From the viewpoint of enabling simple preparation of the ink composition, the use of a substance that exhibits both a function as a dopant and a function as a polymerization initiator is preferred. Examples of such substances include the ionic compounds described above.

### [Additives]

The ink composition may also contain additives as optional components. Examples of these additives include polymerization inhibitors, stabilizers, thickeners, gelling agents, flame retardants, antioxidants, reduction inhibitors, oxidizing agents, reducing agents, surface modifiers, emulsifiers, antifoaming agents, dispersants and surfactants.

### [Contents]

The amount of the solvent in the ink composition can be determined with due consideration of the use of the composition in various application methods. For example, the amount of the solvent is preferably an amount that yields a ratio of the charge transport polymer relative to the solvent that is at least 0.1% by mass, more preferably at least 0.2% by mass, and even more preferably 0.5% by mass or greater. Further, the amount of the solvent is preferably an amount that yields a ratio of the charge transport polymer relative to the solvent that is not more than 20% by mass, more preferably not more than 15% by mass, and even more preferably 10% by mass or less.

### <Organic Layer>

In one embodiment, an organic layer is a layer formed using the charge transport material or the ink composition of an embodiment described above. By using the ink composition, an organic layer can be formed favorably by a coating method. Examples of the coating method include conventional methods such as spin coating methods, casting methods, dipping methods, plate-based printing methods such as relief printing, intaglio printing, offset printing, lithographic printing, relief reversal offset printing, screen printing and gravure printing, and plateless printing methods such as inkjet methods. When the organic layer is formed by a coating method, the organic layer (coating layer) obtained following coating may be dried using a hotplate or an oven to remove the solvent.

In those cases where the charge transport polymer has a polymerizable functional group, the charge transport polymer can be subjected to a polymerization reaction by performing light irradiation or a heat treatment or the like, thereby changing the solubility of the organic layer. By stacking organic layers having changed solubility levels, multilayering of an organic electronic element can be performed with ease. Reference may also be made to WO 2010/140553 in relation to the method used for forming the organic layer.

From the viewpoint of improving the efficiency of charge transport, the thickness of the organic layer obtained following drying or curing is preferably at least 0.1 nm, more preferably at least 1 nm, and even more preferably 3 nm or greater. Further, from the viewpoint of reducing the electrical resistance, the thickness of the organic layer is preferably not more than 300 nm, more preferably not more than 200 nm, and even more preferably 100 nm or less.

### <Organic Electronic Element>

In one embodiment, an organic electronic element has at least one organic layer of the embodiment described above. Examples of the organic electronic element include an organic EL element, an organic photoelectric conversion element, and an organic transistor. The organic electronic element preferably has at least a structure in which an organic layer is disposed between a pair of electrodes.

### [Organic EL Element]

In one embodiment, an organic EL element has at least an organic layer of the embodiment described above. The organic EL element typically includes a light-emitting layer, an anode, a cathode and a substrate, and if necessary, may also have other functional layers such as a hole injection layer, electron injection layer, hole transport layer and electron transport layer. Each layer may be formed by a vapor deposition method or by a coating method. The organic EL element preferably has the organic layer as the light-emitting layer or as another functional layer, more preferably has the organic layer as a functional layer, and even more preferably has the organic layer as at least one of a hole injection layer and a hole transport layer. In one embodiment, formation of the organic layer can be performed favorably by a coating method using the ink composition described above.

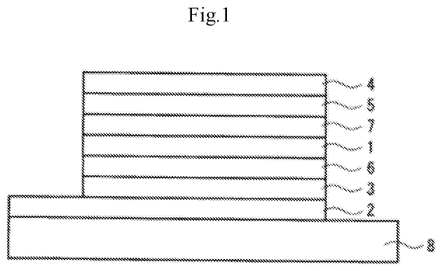

FIG. 1 is a cross-sectional schematic view illustrating one embodiment of the organic EL element. The organic EL element in FIG. 1 is an element with a multilayer structure, and has a substrate 8, an anode 2, a hole injection layer 3, a hole transport layer 6, a light-emitting layer 1, an electron transport layer 7, an electron injection layer 5 and a cathode 4 provided in that order. In one embodiment, at least one of the hole injection layer 3 and the hole transport layer 6 is preferably formed from an organic layer of the embodiment described above. Each of these layers that constitutes the organic EL element is described below in detail.

### [Light-Emitting Layer]

Examples of the materials that can be used for the light-emitting layer include low-molecular weight compounds, polymers, and dendrimers and the like. Polymers exhibit good solubility in solvents, meaning they are suitable for coating methods, and are consequently preferred. Examples of the light-emitting material include luminescent materials, phosphorescent materials, and thermally activated delayed fluorescent materials (TADF).

Specific examples of the luminescent materials include low-molecular weight compounds such as perylene, coumarin, rubrene, quinacridone, stilbene, color laser dyes, aluminum complexes, and derivatives of these compounds; polymers such as polyfluorene, polyphenylene, polyphenylenevinylene, polyvinylcarbazole, fluorene-benzothiadiazole copolymers, fluorene-triphenylamine copolymers, and derivatives of these compounds; and mixtures of the above materials.

Examples of materials that can be used as the phosphorescent materials include meal complexes and the like containing a metal such as Ir or Pt or the like. Specific examples of Ir complexes include FIr(pic) (iridium(III) bis[(4,6-difluorophenyl)-pyridinato-N,C²]picolinate) which emits blue light, Ir(ppy)₃ (fac-tris(2-phenylpyridine)iridium) which emits green light, and (btp)₂Ir(acac) (bis[2-(2′-benzo[4,5-α]thienyl)pyridinato-N,C³]iridium(acetyl-acetonate)) and Ir(piq)₃ (tris(1-phenylisoqionoline)iridium) which emit red light. Specific examples of Pt complexes include PtOEP (2,3,7,8,12,13,17,18-octaethyl-21H,23H-porphyrin-platinum) which emits red light.

When the light-emitting layer contains a phosphorescent material, a host material is preferably also included in addition to the phosphorescent material. Low-molecular weight compounds, polymers, and dendrimers can be used as this host material. Examples of the low-molecular weight compounds include CBP (4,4′-bis(carbazol-9-yl)-biphenyl), mCP (1,3-bis(9-carbazolyl)benzene), CDBP (4,4′-bis(carbazol-9-yl)-2,2′-dimethylbiphenyl), and derivatives of these compounds, whereas examples of the polymers include the charge transport material of the embodiment described above, polyvinylcarbazole, polyphenylene, polyfluorene, and derivatives of these polymers.

Examples of the thermally activated delayed fluorescent materials include the compounds disclosed in Adv. Mater., 21, 4802-4906 (2009); Appl. Phys. Lett., 98, 083302 (2011); Chem. Comm., 48, 9580 (2012); Appl. Phys. Lett., 101, 093306 (2012); J. Am. Chem. Soc., 134, 14706 (2012); Chem. Comm., 48, 11392 (2012); Nature, 492, 234 (2012); Adv. Mater., 25, 3319 (2013); J. Phys. Chem. A, 117, 5607 (2013); Phys. Chem. Chem. Phys., 15, 15850 (2013); Chem. Comm., 49, 10385 (2013); and Chem. Lett., 43, 319 (2014) and the like.

### [Hole Transport Layer, Hole Injection Layer]

Examples of materials that can be used for forming at least one layer selected from the group consisting of hole transport layers and hole injection layers include the charge transport material of the embodiment described above. In one embodiment, at least one of a hole injection layer and a hole transport layer is preferably formed from the charge transport material of the embodiment described above, and it is even more preferable that at least a hole injection layer is formed from the charge transport material of the embodiment described above. For example, in those cases where the organic EL element has an organic layer formed using the charge transport material described above as a hole injection layer, and also has a hole transport layer, a conventional material may be used for the hole transport layer. Further, in those cases where the organic EL element has an organic layer formed using the charge transport material described above as a hole transport layer, and also has a hole injection layer, a conventional material may be used for the hole injection layer.

Examples of conventional materials that can be used for the hole injection layer and the hole transport layer include aromatic amine-based compounds (for example, aromatic diamines such as N,N-di(naphthalen-1-yl)-N,N-diphenyl-benzidine (α-NPD)), phthalocyanine-based compounds, and thiophene-based compounds (for example, thiophene-based conductive polymers (such as poly(3,4-ethylenedioxythiophene):poly(4-styrenesulfonate) (PEDOT:PSS) and the like).

### [Electron Transport Layer, Electron Injection Layer]

Examples of the materials used in electron transport layers and electron injection layers include phenanthroline derivatives, bipyridine derivatives, nitro-substituted fluorene derivatives, diphenylquinone derivatives, thiopyran dioxide derivatives, tetracarboxylic acid anhydrides of condensed-ring such as naphthalene and perylene and the like, carbodiimides, fluorenylidenemethane derivatives, anthraquinodimethane and anthrone derivatives, oxadiazole derivatives, thiadiazole derivatives, benzimidazole derivatives, quinoxaline derivatives, and aluminum complexes. Further, the charge transport material of the embodiment described above may also be used.

### [Cathode]

Examples of the cathode material include metals or metal alloys, such as Li, Ca, Mg, Al, In, Cs, Ba, Mg/Ag, LiF and CsF.

### [Anode]

Metals (for example, Au) or other materials having conductivity can be used as the anode. Examples of the other materials include oxides (for example, ITO: indium oxide/tin oxide, and conductive polymers (for example, polythiophene-polystyrene sulfonate mixtures (PEDOT:PSS)).

### [Substrate]

Glass and plastics and the like can be used as the substrate. The substrate is preferably transparent, and preferably has flexibility. Quartz glass and light-transmitting resin films and the like can be used particularly favorably.

Examples of the resin films include films composed of polyethylene terephthalate, polyethylene naphthalate, polyethersulfone, polyetherimide, polyetheretherketone, polyphenylene sulfide, polyarylate, polyimide, polycarbonate, cellulose triacetate or cellulose acetate propionate.

In those cases where a resin film is used, an inorganic substance such as silicon oxide or silicon nitride may be coated onto the resin film to inhibit the transmission of water vapor and oxygen and the like.

### [Emission Color]

There are no particular limitations on the color of the light emission from the organic EL element. White organic EL elements can be used for various lighting fixtures, including domestic lighting, in-vehicle lighting, watches and liquid crystal backlights, and are consequently preferred.

The method used for forming a white organic EL element may involve using a plurality of light-emitting materials to emit a plurality of colors simultaneously, and then mixing the emitted colors to obtain a white light emission. There are no particular limitations on the combination of the plurality of emission colors, and examples include combinations that include three maximum emission wavelengths for blue, green and red, and combinations that include two maximum emission wavelengths for blue and yellow, or for yellowish green and orange or the like. Control of the emission color can be achieved by appropriate adjustment of the types and amounts of the light-emitting materials.

### <Display Element, Lighting Device, Display Device>

In one embodiment, a display element contains the organic EL element of the embodiment described above. For example, by using the organic EL element as the element corresponding with each color pixel of red, green and blue (RGB), a color display element can be obtained. Examples of the image formation method include a simple matrix in which organic EL elements arrayed in a panel are driven directly by an electrode arranged in a matrix, and an active matrix in which a thin-film transistor is positioned on, and drives, each element.

Furthermore, in one embodiment, a lighting device contains the organic EL element of the embodiment described above. Moreover, in one embodiment, a display device contains the lighting device and a liquid crystal element as a display unit. For example, the display device may be formed as a device that uses the lighting device of the embodiment described above as a backlight, and uses a conventional liquid crystal element as the display unit, namely a liquid crystal display device.

### EXAMPLES

The present invention is described below in further detail using a series of examples, but the present invention is not limited by the following examples.

### <1> Preparation of Charge Transport Polymers

### (Preparation of Pd Catalyst)

In a glove box under a nitrogen atmosphere and at room temperature, tris(dibenzylideneacetone)dipalladium (73.2 mg, 80 μmop was weighed into a sample tube, anisole (15 mL) was added, and the resulting mixture was agitated for 30 minutes. In a similar manner, tris(t-butyl)phosphine (129.6 mg, 640 μmop was weighed into a sample tube, anisole (5 mL) was added, and the resulting mixture was agitated for 5 minutes. The two solutions were then mixed together and stirred for 30 minutes at room temperature to obtain a catalyst solution. In the catalyst preparation, all the solvents used were deaerated by nitrogen bubbling for at least 30 minutes prior to use.

### (Preparation Example 1) Charge Transport Polymer 1

A three-neck round-bottom flask was charged with a monomer 1 shown below (4.0 mmol), a monomer 2 shown below (5.0 mmol), a monomer 3 shown below (2.0 mmol) and anisole (20 mL), and the prepared Pd catalyst solution (7.5 mL) was then added and stirred. After stirring for 30 minutes, a 10% aqueous solution of tetraethylammonium hydroxide (20 mL) was added. The resulting mixture was heated and refluxed for 2 hours. All the operations up to this point were conducted under a stream of nitrogen. Further, all of the solvents were deaerated by nitrogen bubbling for at least 30 minutes prior to use.

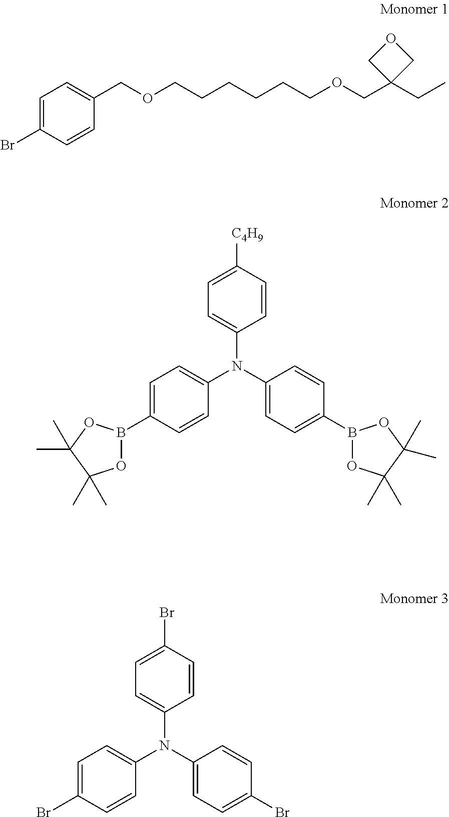

After completion of the reaction, the organic layer was washed with water. The organic layer was then poured into methanol-water (9:1). The resulting precipitate was collected by filtration under reduced pressure, and washed with methanol-water (9:1). The washed precipitate was dissolved in toluene, and re-precipitated from methanol. The thus obtained precipitate was collected by filtration under reduced pressure and then dissolved in toluene, and “Triphenylphosphine, polymer-bound on styrene-divinylbenzene copolymer” (manufactured by Strem Chemicals Inc., 200 mg per 100 mg of the polymer, hereafter referred to as a “metal adsorbent”) was then added to the solution and stirred overnight.

Following completion of the stirring, the metal adsorbent and other insoluble matter were removed by filtration, and the filtrate was concentrated using a rotary evaporator. The concentrate was dissolved in toluene, and then re-precipitated from methanol-acetone (8:3). The thus produced precipitate was collected by filtration under reduced pressure and washed with methanol-acetone (8:3).

The thus obtained precipitate was then dried under vacuum to obtain a charge transport polymer 1.

The thus obtained charge transport polymer 1 had a number average molecular of 7,800 and a weight average molecular weight of 31,000. The charge transport polymer 1 contained a trivalent or higher structural unit B2 (derived from the monomer 3), a divalent structural unit L2 (derived from the monomer 2) and a monovalent structural unit T2 (derived from the monomer 1), and the proportions of those structural units were, in order, 18.2%, 45.5% and 36.4% respectively.

The number average molecular weight and the weight average molecular weight were measured by GPC (relative to polystyrene standards) using tetrahydrofuran (THF) as the eluent. The measurement conditions were as follows.

Feed pump: L-6050, manufactured by Hitachi High-Technologies Corporation

UV-Vis detector: L-3000, manufactured by Hitachi High-Technologies Corporation

Columns: Gelpack (a registered trademark) GL-A160S/GL-A150S, manufactured by Hitachi Chemical Co., Ltd.

Eluent: THF (for HPLC, stabilizer-free), manufactured by Wako Pure Chemical Industries, Ltd.

Flow rate: 1 mL/min

Column temperature: room temperature

Molecular weight standards: standard polystyrenes

### (Preparation Example 2) Charge Transport Polymer 2

A three-neck round-bottom flask was charged with the monomer 2 (5.0 mmol) and the monomer 3 (2.0 mmol) mentioned above in Preparation Example 1, a monomer 4 shown below (4.0 mmol) and anisole (20 mL), and the prepared Pd catalyst solution (7.5 mL) was then added and stirred. Thereafter, the same method as that described for Preparation Example 1 was used to prepare a charge transport polymer 2.

The thus obtained charge transport polymer 2 had a number average molecular of 22,900 and a weight average molecular weight of 169,000. The charge transport polymer 2 contained a trivalent or higher structural unit B2 (derived from the monomer 3), a divalent structural unit L2 (derived from the monomer 2) and a monovalent structural unit T2 (derived from the monomer 4), and the proportions of those structural units were, in order, 18.2%, 45.5% and 36.4% respectively.

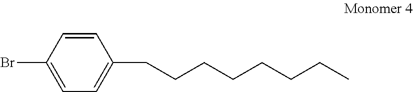

### (Preparation Example 3) Charge Transport Polymer 3

A three-neck round-bottom flask was charged with the monomer 2 mentioned above in Preparation Example 1 (5.0 mmol), the monomer 4 mentioned above in Preparation Example 2 (4.0 mmol), a monomer 5 shown below (2.0 mmol) and anisole (20 mL), and the prepared Pd catalyst solution (7.5 mL) was then added and stirred. Thereafter, the same method as that described for Preparation Example 1 was used to prepare a charge transport polymer 3.

The thus obtained charge transport polymer 3 had a number average molecular of 6,300 and a weight average molecular weight of 50,600. The charge transport polymer 3 contained a trivalent structural unit B1 (derived from the monomer 5), a divalent structural unit L2 (derived from the monomer 2) and a monovalent structural unit T2 (derived from the monomer 4), and the proportions of those structural units were, in order, 18.2%, 45.5% and 36.4% respectively.

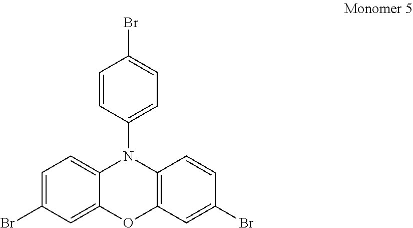

### (Preparation Example 4) Charge Transport Polymer 4

With the exception of using a monomer 6 shown below instead of the monomer 2, a charge transport polymer 4 was prepared using the same method as Preparation Example 3.

The thus obtained charge transport polymer 4 had a number average molecular of 4,300 and a weight average molecular weight of 30,900. The charge transport polymer 4 contained a trivalent structural unit B1 (derived from the monomer 5), a divalent structural unit L2 (derived from the monomer 6) and a monovalent structural unit T2 (derived from the monomer 4), and the proportions of those structural units were, in order, 18.2%, 45.5% and 36.4% respectively.

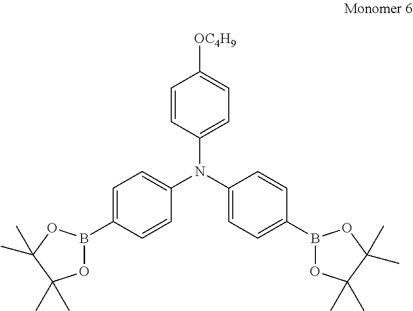

### (Preparation Example 5) Charge Transport Polymer 5

With the exception of replacing the monomer 4 (4.0 mmol) with a combination of the monomer 4 (2.0 mmol) and the monomer 1 (2.0 mmol), a charge transport polymer 5 was prepared using the same method as Preparation Example 3.

The thus obtained charge transport polymer 5 had a number average molecular of 6,500 and a weight average molecular weight of 55,900. The charge transport polymer 5 contained a trivalent structural unit B1 (derived from the monomer 5), a divalent structural unit L2 (derived from the monomer 2), a monovalent structural unit T2 (derived from the monomer 4) and a monovalent structural unit T2 having a polymerizable functional group (derived from the monomer 1), and the proportions of those structural units were, in order, 18.2%, 45.5%, 18.2% and 18.2% respectively.

### (Preparation Example 6) Charge Transport Polymer 6

A three-neck round-bottom flask was charged with the monomer 2 mentioned above in Preparation Example 1 (5.0 mmol), the monomer 4 mentioned above in Preparation Example 2 (2.0 mmol), a monomer 7 shown below (4.0 mmol) and anisole (20 mL), and the prepared Pd catalyst solution (7.5 mL) was then added and stirred. Thereafter, the same method as that described for Preparation Example 1 was used to prepare a charge transport polymer 6.

The thus obtained charge transport polymer 6 had a number average molecular of 5,500 and a weight average molecular weight of 8,700. The charge transport polymer 6 contained a divalent structural unit L1 (derived from the monomer 7), a divalent structural unit L2 (derived from the monomer 2) and a monovalent structural unit T2 (derived from the monomer 4), and the proportions of those structural units were, in order, 36.4%, 45.5% and 18.2% respectively.

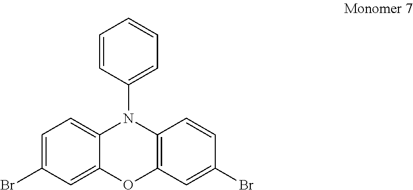

### (Preparation Example 7) Charge Transport Polymer 7

With the exceptions of replacing the monomer 5 (2.0 mmol) with a combination of the monomer 5 (0.75 mmol) and the monomer 7 (2.3 mmol), and using 4.5 mmol and 2.3 mmol respectively of the monomer 2 and the monomer 4, a charge transport polymer 7 was prepared using the same method as Preparation Example 3.

The thus obtained charge transport polymer 7 had a number average molecular of 6,300 and a weight average molecular weight of 47,200. The charge transport polymer 7 contained a trivalent structural unit B1 (derived from the monomer 5), a divalent structural unit L1 (derived from the monomer 7), a divalent structural unit L2 (derived from the monomer 2) and a monovalent structural unit T2 (derived from the monomer 4), and the proportions of those structural units were, in order, 7.7%, 23.1%, 46.2% and 23.1% respectively.

### (Preparation Example 8) Charge Transport Polymer 8

With the exception of using a monomer 8 instead of the monomer 5, a charge transport polymer 8 was prepared using the same method as Preparation Example 3.

The thus obtained charge transport polymer 8 had a number average molecular of 5,300 and a weight average molecular weight of 33,700. The charge transport polymer 8 contained a trivalent structural unit B1 (derived from the monomer 8), a divalent structural unit L2 (derived from the monomer 2) and a monovalent structural unit T2 (derived from the monomer 4), and the proportions of those structural units were, in order, 18.2%, 45.5% and 36.4% respectively.

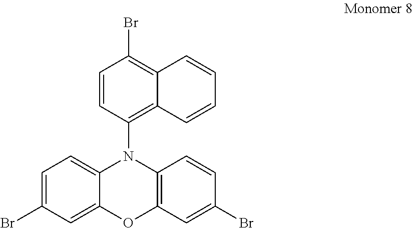

### <2-1> Production of Organic EL Elements

### Example 1

An ink composition 1 was prepared from the charge transport polymer 3 (10.0 mg) obtained in the charge transport polymer synthesis described above, an ionic compound shown below (0.5 mg), and toluene (2.3 mL). Under a nitrogen atmosphere, this ink composition was spin-coated at 3,000 min⁻¹ onto a glass substrate on which ITO had been patterned with a width of 1.6 mm, and the ink composition was then heated on a hotplate at 220° C. for 10 minutes, thus forming a hole injection layer (30 nm).

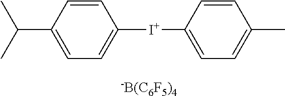 [SMILES: "-B(C6F5)4" ; Cc1ccc([I+]c2ccc(C(C)C)cc2)cc1]

Subsequently, an ink composition 2 was prepared from the charge transport polymer 2 prepared above (20 mg) and toluene (2.3 mL). The ink composition 2 was spin-coated at 3,000 min⁻¹ onto the hole injection layer obtained in the above operation, and the ink composition was then dried by heating on a hotplate at 180° C. for 10 minutes, thus forming a hole transport layer (40 nm).

The thus obtained substrate was transferred into a vacuum deposition apparatus, layers of CBP:Ir(ppy)₃ (94:6, 30 nm), BAlq (10 nm), Alq₃ (30 nm), LiF (0.8 nm) and Al (100 nm) were deposited in that order using deposition methods on top of the hole transport layer, and an encapsulation treatment was then performed to complete production of an organic EL element.

### Example 2

An ink composition 3 was prepared by using the charge transport polymer 4 instead of the charge transport polymer 3 in the ink composition 1 used for forming the hole injection layer in the organic EL element in Example 1. With the exception of forming the hole injection layer using this ink composition 3, an organic EL element was produced in the same manner as Example 1.

### Example 3

An ink composition 4 was prepared by using the charge transport polymer 5 instead of the charge transport polymer 3 in the ink composition 1 used for forming the hole injection layer in the organic EL element in Example 1. With the exception of forming the hole injection layer using this ink composition 4, an organic EL element was produced in the same manner as Example 1.

### Example 4

An ink composition 5 was prepared by using the charge transport polymer 6 instead of the charge transport polymer 3 in the ink composition 1 used for forming the hole injection layer in the organic EL element in Example 1. With the exception of forming the hole injection layer using this ink composition 5, an organic EL element was produced in the same manner as Example 1.

### Example 5

An ink composition 6 was prepared by using the charge transport polymer 7 instead of the charge transport polymer 3 in the ink composition 1 used for forming the hole injection layer in the organic EL element in Example 1. With the exception of forming the hole injection layer using this ink composition 6, an organic EL element was produced in the same manner as Example 1.

### Example 6

An ink composition 7 was prepared by using the charge transport polymer 8 instead of the charge transport polymer 3 in the ink composition 1 used for forming the hole injection layer in the organic EL element in Example 1. With the exception of forming the hole injection layer using this ink composition 7, an organic EL element was produced in the same manner as Example 1.

### Comparative Example 1

An ink composition 8 was prepared by using the charge transport polymer 1 instead of the charge transport polymer 3 in the ink composition 1 used for forming the hole injection layer in the organic EL element in Example 1. With the exception of forming the hole injection layer using this ink composition 8, an organic EL element was produced in the same manner as Example 1.

### <2-2> Evaluation of Organic EL Elements

When a voltage was applied to each of the organic EL elements obtained in Examples 1 to 6 and Comparative Example 1, green light emission was confirmed in each case. For each element, the drive voltage and the emission efficiency at an emission luminance of 1,000 cd/m², and the emission lifespan (luminance half-life) when the initial luminance was 3,000 cd/m² were measured. The results of those measurements are shown in Table 1.

TABLE 1

| | Drive | Emission | Emission |
|---|---|---|---|
| | voltage | efficiency | lifespan |
| | (V) | (cd/A) | (h) |
| Example 1 | 8.0 | 19.0 | 154 |
| Example 2 | 8.2 | 19.3 | 156 |
| Example 3 | 8.3 | 20.1 | 160 |
| Example 4 | 8.1 | 19.1 | 158 |
| Example 5 | 8.0 | 18.0 | 156 |
| Example 6 | 7.9 | 19.6 | 152 |
| Comparative | 8.5 | 17.2 | 145 |
| Example 1 | | | |

As shown in Table 1, the organic EL elements of Examples 1 to 6 had a lower drive voltage, superior emission efficiency and a longer emission lifespan than the element of Comparative Example 1. In other words, in terms of the constituent material for the hole injection layer, it is evident that by using a charge transport polymer having a structural unit containing an N-aryl phenoxazine skeleton within the molecule as the charge transport material, effects including a reduction in the drive voltage and improvements in the emission efficiency and the emission lifespan can be achieved.

The effects of the embodiments of the present invention have been indicated by the examples described above. However, the present invention is not limited to the charge transport polymers used in the examples, and similar organic electronic elements can be obtained even when other charge transport polymers are used, provided those other charge transport polymers remain within the scope of the present invention. Further, in the thus obtained organic electronic elements, excellent characteristics similar to those obtained in each of the above examples can be achieved.

### DESCRIPTION OF THE REFERENCE SIGNS

1: Light-emitting layer
2: Anode
3: Hole injection layer
4: Cathode
5: Electron injection layer
6: Hole transport layer
7: Electron transport layer
8: Substrate

1. A charge transport material comprising a charge transport polymer, wherein the charge transport polymer contains a structural unit having an N-aryl phenoxazine skeleton.

2. The charge transport material according to claim 1, wherein the structural unit having an N-aryl phenoxazine skeleton comprises at least one structural unit selected from the group consisting of divalent structural units L1 and trivalent or higher structural units B1.

3. The charge transport material according to claim 1, wherein the charge transport polymer also contains at least one structural unit, besides the structural unit having an N-aryl phenoxazine skeleton, selected from the group consisting of divalent structural units L2 having charge transport properties and trivalent or higher structural units B2 having charge transport properties.

4. The charge transport material according to claim 1, wherein the charge transport polymer also contains a divalent structural unit L2 having charge transport properties besides the structural unit having an N-aryl phenoxazine skeleton, and
the divalent structural unit L2 having charge transport properties contains at least one structure selected from the group consisting of aromatic amine structures, carbazole structures, thiophene structures, benzene structures and fluorene structures.

5. The charge transport material according to claim 1, wherein the charge transport polymer has a structure that branches in three or more directions.

6. The charge transport material according to claim 1, wherein the charge transport material is used as a hole injection material.

7. An ink composition comprising the charge transport material according to claim 1.

8. An organic electronic element having an organic layer formed using the charge transport material according to claim 1.

9. An organic electroluminescent element having an organic layer formed using the charge transport material according to claim 1.

10. The organic electroluminescent element according to claim 9, also having a flexible substrate.

11. The organic electroluminescent element according to claim 10, wherein the flexible substrate comprises a resin film.

12. A display element having the organic electroluminescent element according to claim 9.

13. A lighting device having the organic electroluminescent element according to claim 9.

14. A display device having the lighting device according to claim 13, and a liquid crystal element as a display unit.
